(19) United States
(12) Patent Application Publication
(10) Pub. No.: US 2015/0190796 A1
(43) Pub. Date: Jul. 9, 2015

(54) Composite Material Consisting of a Catalyst/Phase-Change Material, Related Manufacturing Methods and Use of Such a Material in Catalytic Reactions

(71) Applicant: COMMISSARIAT A L'ENERGIE ATOMIQUE ET AUX ENERGIES ALTERNATIVES, Paris (FR)

(72) Inventors: Laurent Bedel, Quaix En Chartreuse (FR); Jerome Gavillet, Saint-Egreve (FR)

(73) Assignee: COMMISSARIAT A L'ENERGIE ATOMIQUE ET AUX ENERGIES ALTERNATIVES, Paris (FR)

(21) Appl. No.: 14/415,462

(22) PCT Filed: Jul. 17, 2013
(86) PCT No.: PCT/IB2013/055861

§ 371 Date: Jan. 16, 2015

(30) Foreign Application Priority Data
Jul. 17, 2012 (FR) 1256881

(51) Int. Cl. B01J 31/26; B01J 35/02; B01J 37/34; B01J 37/02; C07C 29/152; C07C 1/12; C07C 1/04; B01J 31/02; C07C 29/157

(52) U.S. Cl. CPC B01J 31/26; B01J 31/0202; B01J 35/026; B01J 37/347; C07C 29/157; C07C 29/152; C07C 1/12; C07C 1/0435; C07C 1/0445; C07C 1/0415; B01J 37/0221; B01J 2531/002; B01J 2531/005; B01J 2231/005

(57) ABSTRACT

Material with hybrid particles (1) each consisting of a particle (2) of a phase-change material (PCM) interfaced with a catalytic material (3) in solid form, the size of the hybrid particles being between 0.1 mm and 10 mm, preferably between 1 mm and 5 mm.

### FIELD OF TECHNOLOGY

The present invention relates to the field of exothermic or endothermic chemical reactions by heterogeneous catalysis.

The present invention relates more particularly to the development of a new material with hybrid particles comprising a phase that catalyzes these reactions.

The invention also relates to the use of such a new material and the related manufacturing methods.

The invention is implemented advantageously in a continuous reaction in a reaction chamber of a reactor of the circulating fluidized-bed type.

### PRIOR ART

Exothermic catalytic reactions, such as the synthesis of methane, methanol from synthesis gas commonly called syngas, or endothermic reactions such as dehydrogenation or reforming reactions require precise control of the temperature in the synthesis reactor to guarantee an optimal yield and to avoid premature deactivation of the catalytic phase. In the case of exothermic reactions, the energy of the reaction must be removed from the reaction chamber, and vice-versa for the endothermic reactions.

In particular, the reactions of hydrogenation of CO or of CO₂ to methane or methanol are exothermic and reactor design is known to be complex as it is necessary to integrate heat exchangers in the reaction zone, i.e. at least along the reaction chamber. We may mention here the process for converting natural gas to methanol operated under the trade name “Lurgi Megamethanol® process”. Moreover, these reactions take place in the presence of catalysts and any harmful thermal runaway of a reactor is likely to accelerate catalyst aging and deactivation, cause a loss of process productivity and efficiency and in some circumstances lead to thermal runaway if the heat of the chemical reaction cannot be removed effectively and sufficiently quickly from the reaction chamber.

Moreover, it is known that phase-change materials (PCMs) are materials capable of displaying a reversible physical phase change whose associated change of enthalpy (or latent heat) allows storage and draw-down of thermal energy. In terms of spatial density, the storage capacity of a PCM material is typically 3 to 4 times greater than it is possible to reach with sensible heat. The phase change of a PCM material is of an isothermal nature, i.e. it takes place at constant temperature.

There are four types of phase change for PCM materials, respectively solid-solid, solid-liquid, solid-vapor and liquid-vapor. The latent heat and the volume change of a PCM material are higher the greater the change of order (given by the entropy change) associated with the phase transition. Thus, for example, a solid-vapor change of order is higher than a solid-liquid change of order which in its turn is higher than a solid-solid change of order.

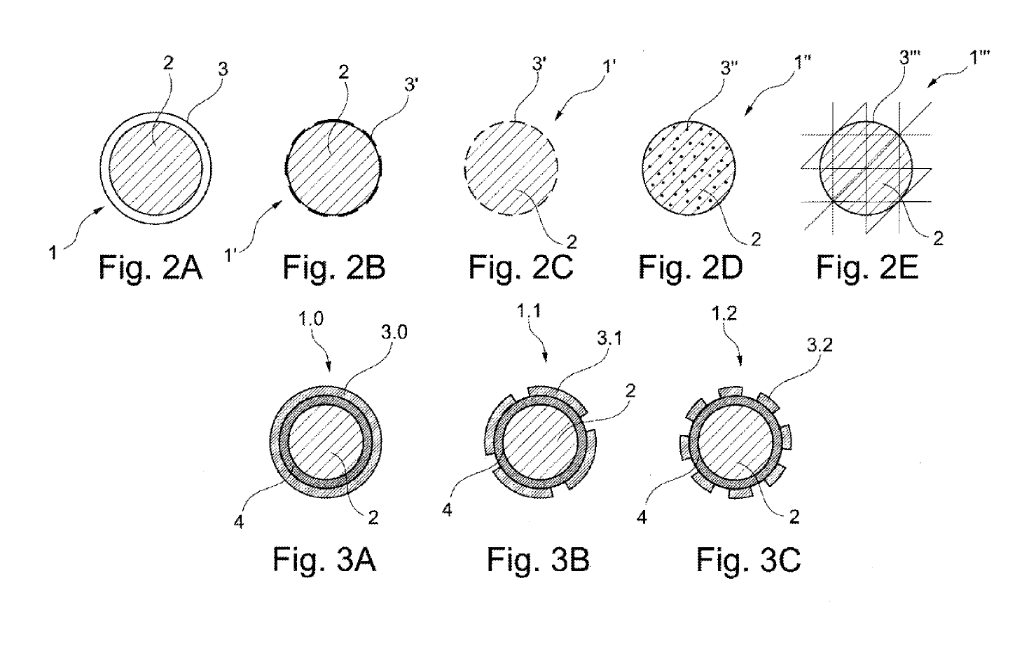

There are several families of PCM materials ranging from simple materials to compound materials: reference may notably be made to FIG. 2 of publication [1]. Among the simple materials, conventionally a distinction is made between organic materials and inorganic materials. Among the compound materials, a distinction is made between eutectic compounds of organic-organic, organic-inorganic and inorganic-inorganic types. These families of PCM materials may also be classified according to the chemical nature of the materials: paraffins, fatty acids, hydrates of salts, nitrates, etc. Reference may be made to publication [2], which illustrates a classification of families of PCM materials as a function of their spatial density of thermal storage.

For each of these materials there is a corresponding unique phase change temperature and a unique spatial density of latent heat. Physically, these two parameters are linked by a linear relation, such that the spatial density of latent heat increases with the phase change temperature.

In practice, a PCM material may be selected as a function of the operating temperature of a system for which the material is intended.

For systems employing chemical reactions, the choice of PCM material is usually made as a function of the temperature at which the chemical reaction takes place and the thermal energy to be stored or released.

More generally, a PCM material may be selected taking into account various criteria, which may be enumerated as follows:
thermodynamic: a suitable phase change temperature, a high latent heat, high thermal conductivity;
intrinsic physical properties: high density, small volume change, reproducibility and stability in cycling;
chemical properties: chemical stability, compatibility with the other materials of the system, toxicity, flammability;
phase change kinetics: no supercooling, high degree of crystallization;
economic: abundant, available, low cost, recyclable.

Publication [3] gives a good summary of the reasons why bulk PCM materials are not very useful for applications of precise temperature control within a zone for chemical synthesis by heterogeneous catalysis. Firstly, for optimal exploitation of the latent heat of fusion of the PCM materials for absorbing heat, it is necessary for these materials to be sufficiently dispersed in order to melt quickly. It has been shown that if the size of the particles is reduced by a factor of 10, the time taken for complete fusion decreases by a factor of about 100. Then, so that it can be used in a chemical synthesis reactor, the PCM material must be encapsulated in a shell with good hermeticity and good stability to prevent poisoning of the catalytic phase. Finally, the PCM material must be distributed uniformly in the reaction mixture so as to avoid hot spots, which may accelerate catalyst deactivation.

Encapsulation of nanoparticles of PCM materials for controlling the temperatures of exothermic heterogeneous reactions has already been proposed: see publication [3].

Patent application US 2008/0272331 also proposes hybrid nanoparticles with a size between 10 and 100 nm, with an envelope of a heat-conducting metallic material encapsulating a PCM. These dimensions make it possible to obtain a high thermal storage power as small particles can melt or solidify rapidly. Nevertheless, they have a low unit storage capacity and consequently a large quantity of these particles is required for efficient storage of the heat produced by the chemical reaction.

Moreover, the use of PCM nanoparticles was explicitly formulated and tested experimentally in the context of fixed-bed catalytic reactions, by mixing PCM powders and powders of catalysts (catalytic materials) [3]. Moreover, the authors note the possibility of loading the catalyst directly on the PCM. However, no concrete embodiment or concept is proposed. Moreover, this vision of the authors is limited to a perfect case of use, since in practice the nanometric size of the particles cannot allow homogeneous distribution in a carrying fluid, nor even in a stationary medium, still less in flow conditions such as in a circulating or noncirculating fluidized bed. This last-mentioned point raises a critical problem, that of uniform dispersion, in space and time, of a large number of fine particles in a stationary or flowing reaction mixture.

There is therefore a need to improve the solutions using a phase-change material PCM in a catalytic chemical reaction so that the latter may store or release the heat produced or avoid any hot spot and thermal runaway in the reaction zone of a reactor.

There is a particular need for a new solution using a phase-change material PCM in a catalytic chemical reaction, which should be simple to implement and of lower cost.

### SUMMARY OF THE INVENTION

For this purpose, the invention relates, in one of its aspects, to a material with hybrid particles each constituted of a particle of a phase-change material (PCM) at the interface with a catalytic material in solid form, the size of the hybrid particles being between 0.1 mm and 10 mm, preferably between 1 mm and 5 mm.

“Catalytic material” means, here and in the context of the invention, the usual sense of the term, i.e. a solid material on which the reaction takes place in at least one step and which makes it possible to lower the energy of activation as well as the temperature of the chemical reaction, without appearing in the equation of the reaction.

“Interfaced with” means, here and in the context of the invention, that the PCM material and the catalytic material are in direct physical contact without using a specific intermediate material for achieving their adherence or in indirect physical contact by means of an interface material required for encapsulation of the PCM material when the latter displays a liquid phase.

It is to be noted here that a PCM material displaying a vapor phase or a high vapor pressure is excluded from the PCM materials that may be suitable for carrying out the invention. To date, to the best knowledge of the inventors, a great majority of PCM materials have a low vapor pressure and may therefore be suitable for carrying out the invention. Reference may be made notably to publication [4], which describes PCM materials intended for an application of thermal management in buildings, and which have this characteristic

Thus, the invention consists essentially of proposing hybrid particles of materials including a catalytic material intended for catalyzing an exothermic or endothermic chemical reaction, which is deposited on the PCM material, which for its part will store or respectively release the thermal energy derived from said chemical reaction. Thus, by means of the same hybrid particles according to the invention, we achieve both the catalysis of a chemical reaction and suppression of heating and if applicable thermal runaway (in the case of an exothermic reaction) of a reactor employing the chemical reaction.

The dimensions according to the invention allow a satisfactory compromise between high thermal storage power, high storage capacity and a sufficient capacity for dispersion of the particles in a fluid, which makes it possible to use the material according to the invention in any catalytic chemical reaction and notably in a fluidized bed. In particular, with hybrid particles larger than 100 μm, thermal energy storage densities at least equal to 10 J/m² are obtained, i.e. energy densities that commonly occur in catalytic chemical reactions.

Advantageously, the PCM material is selected from paraffins, nitride-based eutectic materials, nitrates, hydroxides, fluorides, carbonate, molten salts such as NaNO₂, NaNO₃, NaOH, LiOH, NaCl, metal alloys such as AlSi, capable of containing one or more heat-conducting elements such as carbon nanotubes, metals such as Cu, Al, Si.

More advantageously, the catalytic material covers an area between 1 and 100%, preferably between 10 and 100% of the outer surface of each hybrid particle.

The material with hybrid particles according to the invention may comprise a continuous layer, of a material different than the catalytic material, encapsulating the PCM material. A continuous layer is selected that is flexible so that it can accommodate the volume changes of the PCM material that are caused by the phase changes during a catalytic chemical reaction. The compliance of the encapsulating layer is preferably determined for accommodating volume changes of the PCM reversibly so as to allow the material according to the invention to undergo cycles. This continuous encapsulating layer may have properties of a physical and chemical barrier on the one hand between the PCM material and the catalytic material and on the other hand between the PCM material and the reactants of the catalytic chemical reaction. This encapsulating layer may also have mechanical properties, performing the role of mechanical barrier in order to protect the PCM material against erosion. An encapsulating material is selected with a high coefficient of thermal conductivity, at least equal to that of the PCM material.

According to a first embodiment, the catalytic material is in the form of a continuous layer encapsulating the PCM material. According to this embodiment, the PCM material may display both a solid-solid and a solid-liquid phase change without it being necessary to encapsulate it in an additional material.

According to a second embodiment, the catalytic material is in the form of a discontinuous layer partially covering the PCM material.

According to a third embodiment, the catalytic material is in the form of discrete particles dispersed on the surface or in the volume of each particle of PCM material. According to these second and third embodiments, the PCM material may display both a solid-solid and a solid-liquid phase change but in the latter case it is necessary to encapsulate it in an additional material.

According to a fourth embodiment, the catalytic material is in the form of an open structure in which at least one particle of PCM material is impregnated. According to this embodiment, the PCM material may display both a solid-solid and a solid-liquid phase change.

The catalytic material may be composed partly or completely with: Cu, Zn, Al, Cr, Ce, Zr, Pt, Pd, Ni, Ti, Si, and the corresponding oxides and nitrides.

The invention also relates, in another of its aspects, to the use of the material with hybrid particles that has just been described in an exothermic or endothermic catalytic reaction.

The exothermic catalytic reaction may be a hydrogenation reaction. It may advantageously be the synthesis of methane (CH₄) by hydrogenation of carbon dioxide (CO₂), the catalytic material being Ni—Al₂O₃. It may also advantageously be the synthesis of methanol from synthesis gas (CO+CO₂+H₂), the catalytic material being Cu/ZnO/Al₂O₃.

Preferably, the reaction is carried out continuously in a reaction chamber of a reactor in which the hybrid particles are circulated.

The reactor is advantageously a reactor of the circulating fluidized-bed type.

The invention also relates, in another of its aspects, to a powder of material with hybrid particles described above.

The invention also relates, in another of its aspects, to a first method of manufacturing a material with hybrid particles each consisting of a particle of a phase-change material (PCM) interfaced with a catalytic material in solid form, according to which the following steps are carried out:
a/ production of a PCM material in powder form;
b/ deposition of a thin layer of a catalytic material continuously or discontinuously on the particles of the PCM material.

According to this first method, step b/ may be carried out either by physical vapor deposition (PVD), by magnetron cathodic sputtering, or by chemical vapor deposition (CVD), or by a sol-gel technique, or by an electrodeposition technique, or by a liquid impregnation technique.

The invention relates to a second method of manufacturing a material with hybrid particles each consisting of a particle of a phase-change material (PCM) interfaced with a catalytic material in solid form, according to which the following steps are carried out:
a1/ production of a PCM material in powder form;
a2/ production of a catalytic material in powder form;
b1/ mixing the powders of catalytic material and PCM material;
c1/ annealing the mixture above the phase change temperature of the PCM material;
d1/ grinding the annealed mixture.

The invention finally relates to a third method of manufacturing a material with hybrid particles each consisting of a particle of a phase-change material (PCM) interfaced with a catalytic material in solid form, according to which the following steps are carried out:
a′1/ production of a PCM material in powder form;
a′2/ production of a catalytic material in the form of an open structure;
b′/ impregnation of the open structure of the catalytic material with the PCM material dissolved or dispersed in an aqueous phase or a solvent;
c′/ annealing the impregnated structure above the phase change temperature of the PCM material and the temperature of evaporation of the water or solvent.

According to one embodiment, once step a/ or all of production of the PCM material in powder form is completed, a step of deposition of a continuous layer, of a material different than the catalytic material encapsulating the PCM material, is carried out.

### DETAILED DESCRIPTION

Other advantages and features of the invention will become clearer on reading the detailed description of examples of carrying out the invention, provided for purposes of illustration and nonlimiting, referring to the following figures, in which:

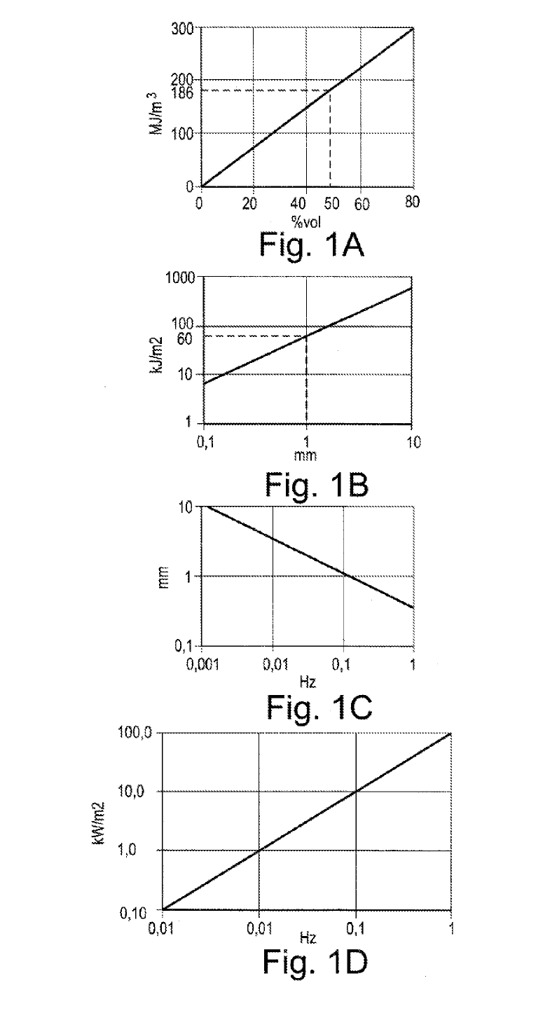

FIGS. 1A to 1D illustrate the various curves of the characteristics of a phase-change material (PCM) already marketed and serving for making a hybrid particle of the material according to the invention;

FIGS. 2A to 2 E are schematic views showing different forms of hybrid particles of the material according to the invention;

FIGS. 3A to 3C are schematic views showing different forms of hybrid particles of the material according to one embodiment of the invention;

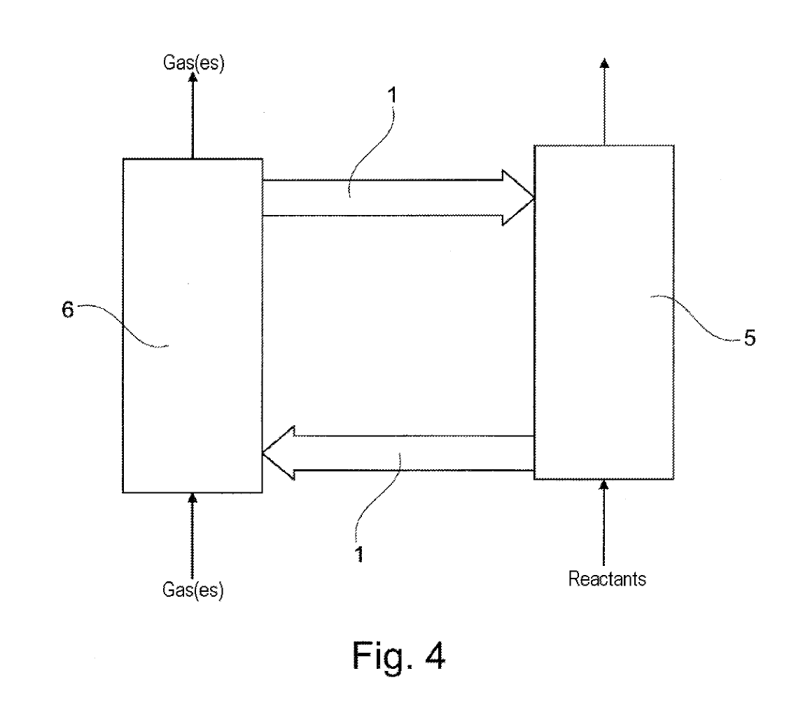

FIG. 4 is a schematic example of continuous synthesis plant using hybrid particles of the material according to the invention.

In all the following examples, an exothermic chemical reaction is considered, carried out by catalysis at a temperature of about 200° C.

The following examples 1 to 5 give different embodiment examples of a material with hybrid particles starting from one and the same phase-change material PCM with change of state between a solid phase and a solid phase, this material already being marketed under the name X180® or “PlusICE X180®” by the company PCM Products.

These materials comprise one or more polyhydric alcohols, such as ribitol with a melting point of 102° C.; fucitol with a melting point of 153° C.; or inositol with a melting point of 226° C. or a mixture thereof.

The material X180® or “PlusICE X180®” is a mixture of these polyhydric alcohols and has a melting point of 180° C.

The table given below shows the intrinsic properties of this material as given by the company PCM Products: see [5].

TABLE

| Tempera- | | | | | | |
|---|---|---|---|---|---|---|
| ture of | | Latent | Spatial | Specific | Volume | Thermal |
| change of | | heat | storage | heat | change | conduc- |
| state | Density | capacity | density | capacity | ΔV | tivity |
| (° C.) | (kg/m³) | (kJ/kg) | (MJ/m³) | (kJ/kg K) | (%) | (W/K · m) |
| 180 | 1.33 | 280 | 372 | 1.40 | 9.0 | 0.360 |

Based on the intrinsic properties of a PCM material, X180®, it is possible to define the characteristics and dimensioning of this material so that it is suitable for the conditions of thermal storage of the exothermic chemical reaction by catalysis.

Thus, it can firstly be seen from the table that the phase change temperature of the PCM material, X180® is 180° C., or 20° C. below the nominal operating temperature of the intended chemical reaction (200° C.). This thermal gradient allows the heat to diffuse from the environment near the PCM material to its surface, then to the fusion front within the PCM material, thus allowing storage of heat during the exothermic reaction.

It also follows from the table that the spatial density of thermal storage of the PCM material, X180® is 372 kJ/m³. Also, the volume concentration of this PCM material in the operational environment of the reaction then allows preliminary definition of the spatial density of thermal storage of the heat from the reaction. FIG. 1A shows the possible spatial density of thermal storage as a function of the volume concentration of the PCM material, X180®. This linear relation is reflected, for example for a volume concentration of PCM of 50%, in a possible spatial density of thermal storage of about 186 kJ/m³.

It is possible to calculate the surface density of storage of thermal energy of the PCM material, X180® as a function of a given thickness of the latter. This linear relation is shown in FIG. 1B. For example, the surface density of storage energy is about 60 kJ/m² for a thickness of PCM material, X180® of the order of 1 mm. By equivalence, this value corresponds to an average diameter in the case when a PCM material is used in powder form, which makes it possible to define a granulometry as a function of the surface density of heat locally available in the reaction chamber carrying out the catalytic exothermic reaction.

To a first approximation, following an analytical approach of a simplified model of PCM material comparable to a semi-infinite medium, the depth of fusion S(t) of the PCM material may be defined as a function of time t according to the following equation:

$$S ( t ) = \sqrt{2 \cdot \frac{λ \cdot Δ T}{L} \cdot t}$$

where S(t) is the position of the phase change front (or depth of thermal penetration) at time point t,
λ is the thermal conductivity of the PCM (W/K.m),
ΔT is the temperature difference between the envelope of the material and the untransformed PCM material (no phase change),
L is the latent heat of phase change of the PCM (J/m³).

Thus, for a given thickness of the PCM material or a given particle diameter (granulometry) of the material in powder form, it is possible to define a time, or in other words a frequency, of complete fusion of the material. FIG. 1C shows the linear relation between the thickness or particle diameter of the PCM and the frequency of fusion. It is thus possible to define an average frequency that is optimal as a function of the conditions of use of the PCM material. For example, for an average particle diameter of powder of 1 mm, the time for fusion to the center of the material is about 8 seconds, i.e. the frequency of fusion is 0.125 Hz.

Consequently, it is possible to calculate the surface density of thermal storage power that the PCM material may display from the energy surface density of thermal storage energy (FIG. 1B) and as a function of the frequency of fusion (FIG. 1C), i.e. as a function of the frequency of fusion in exothermic reaction conditions. FIG. 1D shows the surface density of thermal storage power of the PCM material, X180® as a function of the frequency, it being specified that the average particle diameter of the powder is 1 mm for a temperature difference between the temperature of the exothermic reaction and of phase change of the material of the order of 20° C. Thus, a powder of PCM material, X180® with granulometry of 1 mm can support a storage power density of about 14 kW/m² (about 9 kW/m² of latent heat and 5 kW/m² of sensible heat) at an operating frequency of 0.125 Hz.

In conclusion, the type of PCM, in this case the PCM material, X180®, its morphology and the dimensions of these particles can and should be adapted to the thermal storage conditions required by the exothermic reaction: energy density, power density and thermal load time.

Some examples of production of hybrid particles of a material according to the invention, each particle consisting of a particle of a PCM material X180® with granulometry of 1 mm interfaced with a catalytic material in solid form, are now described, referring to FIGS. 2A to 2 E.

### Examples 1 to 3

Using a technique of physical vapor deposition (PVD), more precisely magnetron cathodic sputtering (“magnetron sputtering”), catalyst material is deposited on each particle of PCM material X180® at room temperature or below the phase change temperature of the PCM.

The particles of PCM material X180® are first positioned on the lower part of a drum with a diameter of the order of 1 m. A target for cathodic sputtering PVD is positioned inside the drum, advantageously a few centimeters above the PCM particles.

After putting the deposition chamber under vacuum, a flow of argon and optionally a reactive gas (O₂ or N₂ or CH₄) are introduced into the deposition chamber and the pressure is adjusted to the deposition pressure, typically to about 1 Pa.

The drum is then rotated in order to ensure uniform deposition on all the PCM particles. Then an electric discharge is applied to the PVD target for a predetermined time.

After stopping the electric discharge on the target, rotation of the drum is stopped.

Finally, the deposition chamber is opened and the PCM particles, each coated with catalytic material, are removed from the chamber.

### Example 1

A PVD target is selected with a composition such as to produce a continuous layer of a catalyst with composition 5 wt % Pd-Alumina.

A weight of about one kilogram of powder of PCM material X180® is put in the drum.

A radio-frequency RF electric discharge is applied to the target with a power density of the order of 10 W.cm² for two hours.

After deposition, the hybrid particles 1 of the material according to the invention are obtained, namely PCM particles 2 covered with a continuous encapsulating layer 3 of about 2 μm of Pd-Alumina. In this example 1, the continuous layer 3 of Pd-Alumina catalytic material therefore also serves as the layer for encapsulating the PCM material.

The possible form of one of these particles is shown in FIG. 2A.

### Example 2

A PVD target is selected with a composition such as to produce a continuous layer of a catalyst with the composition 40 wt % silica-Alumina.

The same steps are followed as in example 1.

After deposition, the hybrid particles 1 of the material according to the invention are obtained, namely PCM particles 2 covered with a continuous catalytic layer 3 of about 2 μm of Silica-Alumina. The form of one of these particles is shown in FIG. 2A.

### Example 3

A silver PVD target is selected, for producing a discontinuous layer of silver.

A weight of about one kilogram of powder of PCM material X180® is put in the drum.

An electric discharge of direct current DC of pulsed type is applied to the target with a power density of the order of 1 W.cm² for 5 minutes.

After deposition, the hybrid particles 1′ of the material according to the invention are obtained, namely PCM particles 2 covered with a discontinuous layer 3′ of silver. The discontinuous layer may consist of particles of silver with a size between 50 and 100 μm.

The possible form of one of these particles 1′ is shown in FIG. 2B or 2C as a function of the size of the silver particles.

### Example 4

A powder of catalyst materials and a powder of PCM material X180® are produced first, in a weight ratio of about 1:10, as a nonlimiting example.

Then the two powders are mixed together.

Then the mixture obtained is annealed above the phase change temperature of the PCM material X180®, i.e. above 180° C.

Finally, grinding of the annealed mixture is carried out.

After grinding, the hybrid particles 1″ of the material according to the invention are obtained, namely PCM particles 2, in the volume of which the particles 3″ of catalyst are dispersed. The possible form of one of these particles is shown in FIG. 2D.

### Example 5

An open structure of catalytic material is produced by the method described in patent application FR 11 59685 or patent application WO2010/012813.

It is to be noted that although these patent applications describe respectively the production of structures with a polymer matrix (open structures) in which nanoparticles are dispersed and three-dimensional open structures of carbon nanotubes or nanofibers in which silicon nanoparticles are uniformly distributed, the production of open structures with catalyst materials according to the invention in place of the silicon is entirely conceivable.

The open structure of catalyst material is then impregnated with the PCM material X180® dissolved or dispersed in an aqueous phase or a solvent.

Finally the impregnated structure is annealed above the phase change temperature of the PCM material X180® and above the temperature of evaporation of the water or solvent, i.e. above 180° C.

After annealing, the hybrid particles 1′ of the material according to the invention are obtained, namely an open structure 3′″ of catalyst in which PCM particles 2 are impregnated. The possible form of one of these particles is shown in FIG. 2 E.

The hybrid particles 1′, 1″, 1′″, the methods of manufacture of which have just been described, may be used advantageously by circulating them continuously in an exothermic or endothermic reaction chamber of a reactor.

Advantageously, the reactor is of the circulating fluidized-bed type (“dual fluidized-bed reactor”).

FIGS. 3A to 3C show one embodiment of the invention: according to this embodiment, the hybrid particles 1.0, 1.1, 1.2, all comprising a continuous layer 4, of material different than the catalytic material, encapsulating the PCM material 2. This continuous layer 4 is flexible so that it can accommodate the volume changes of the PCM material 2, which are induced by the phase changes, during a catalytic chemical reaction. In FIG. 3A, the layer 3.0 of catalyst is also continuous, whereas in FIG. 3B, the layer 3.1 of catalyst is discontinuous and in FIG. 3C, the particles 3.2 of catalyst material are dispersed on the surface of the continuous encapsulating layer 4.

Various methods may be envisaged for performing deposition of the continuous encapsulating layer 4. As an example, the flexible layer 4 may be synthesized by a PECVD technique (“plasma enhanced chemical vapor deposition”) in a fluidized bed as described in articles [6] or [7]. HMDSO (hexamethyldisiloxane) is introduced into the PECVD reactor to form a film of silicon oxide SiOx. The polymeric character, which confers the property of accommodation due to the phase change, of this layer 4 is controlled by the ratio of O₂ to neutral gas (Ar or He) of the gas atmosphere in the reactor. The deposition time and the electric power injected in the plasma are adjusted to form a continuous layer of several microns (from 1 μm to 10 μm).

As another example, a method of deposition by the sol-gel technique may also be envisaged.

FIG. 4 shows the principle of a plant carrying out a continuous exothermic reaction, namely synthesis of methanol, in which the hybrid particles 1 according to the invention circulate concomitantly but in countercurrent to synthesis gas or syngas (CO+CO₂+H₂) in the synthesis reactor 5.

More precisely, the reaction takes place at around 200° C. and the catalyst material constituting the continuous encapsulating layer is Cu/ZnO/Al₂O₃.

Also more precisely, in this plant, the hybrid particles 1 are extracted from a reactor-heat exchanger 6 by means of a cyclone and are sent into the synthesis reactor 5.

In the synthesis reactor 5, the hybrid particles 1 according to the invention circulate in countercurrent to the synthesis gas, which reacts to form methanol.

The hybrid particles 1 according to the invention, whose PCM material 2 undergoes a first phase change in the synthesis reactor, are then injected into a heat exchanger in order to undergo the reverse phase change before being injected back into the synthesis reactor, and so on.

Thus, the cycle that the hybrid particles 1 according to the invention undergo in the plant that has just been described is as follows.

The catalytic exothermic methanol synthesis reaction takes place on the active layer of catalyst 3, the thermal energy of the reaction is stored directly by the PCM material 2 in the synthesis reactor 5, which thus prevents heating and thermal runaway of the latter.

At the outlet of the synthesis reactor 5, the PCM material is in its hottest phase, until the hybrid particle 1 is cooled in the reactor-heat exchanger 6.

At the outlet of the latter, the PCM material 2 has changed phase again, i.e. the particle 1 has a PCM material in its coldest phase and the layer of catalyst 3.

The invention is not limited to the examples that have just been described;

notably, characteristics of the examples illustrated can be combined together in variants that are not illustrated.

Other improvements or variants may be envisaged while remaining within the scope of the invention.

Thus, for example, other methods may be envisaged for depositing the layers of catalyst on the particles of PCM material, notably by techniques of the sol-gel type, of the chemical vapor deposition type (CVD) and of the liquid impregnation type.

Moreover, we may envisage carrying out any catalytic reaction, whether exothermic or endothermic, by means of the hybrid particles according to the invention. Notably, reactions of hydrogenation or of dehydrogenation may be envisaged. Thus, an example of exothermic reaction other than that described above is the synthesis of methane CH₄ by hydrogenation of syngas. This exothermic reaction takes place at about 300° C. with Ni—Al₂O₃ catalyst material.

Moreover, instead of the material X180® or “PlusICE X180®”, we may certainly envisage using other PCM materials. In particular, other materials comprising one or more polyhydric alcohols may be envisaged.

Thus, a material may be envisaged with a melting point equal to 190° C. comprising fucitol and inositol with 50% of each.

Another material may also be envisaged with a melting point equal to 160° C. comprising ribitol, fucitol and inositol with 33% of each.

The expression “comprising a” must be understood as being a synonym of “comprising at least one”, unless stated otherwise.

### REFERENCES CITED

[1]. “Technology of Latent Heat Storage for High Temperature Application: A Review”, T. NOMURA, N. OKINAKA and T. AKIYAMA, ISIJ International, Vol. 50 (2010), No. 9, pp. 1229-1239.
[2]. “Heat & cold storage with MCP: an up to date introduction into basics & applications (Heat & mass transfer)”, MEHLING H., CABEZA L. F. 2008.
[3]. “Encapsulated nano-heat-sinks for thermal management of heterogeneous chemical reactions”, Zhang. M, Hong. Y, Ding. S, Hu. J, Fan. Y, Voevodin. A. A., Su. M, The Royal Society of Chemistry (2010), Nanoscale, 2010, 2, pp. 2790-2797.
[4]. “Phase change material-based building architecture for thermal management in residential and commercial establishments>>, A. Pasupathya, R. Velraja, R. V. Seenirajb, Renewable and Sustainable Energy Reviews 12 (2008) pp. 39-64.
[5]. http://www.pcmproducts.net/files/x_range.pdf. http://www.pcmproducts.net/files/PlusICE %20Range-2013.pdf.
[6]. Sun E Y, Nutt S R, Brennan J J. Surface and Coatings Technology 2003; 169-170:544-548.
[7]. Karches M, von Rohr PR. Surface and Coatings Technology 2001; 142-144:28-33.

1.-16. (canceled)

17. A material with hybrid particles each consisting of a particle of a phase-change material interfaced with a catalytic material in solid form, the size of the hybrid particles being between 0.1 mm and 10 mm.

18. The material with hybrid particles as claimed in claim 17, wherein the size of the hybrid particles is between 1 mm and 5 mm.

19. The material with hybrid particles as claimed in claim 17, the PCM material being selected from paraffins, nitride-based eutectic materials, nitrates, hydroxides, fluorides, carbonate, molten salts such as NaNO₂, NaNO₃, NaOH, LiOH, NaCl, metal alloys such as AlSi, capable of containing one or more heat-conducting elements such as carbon nanotubes, metals such as Cu, Al, Si.

20. The material with hybrid particles as claimed in claim 17, the catalytic material covering an area between 1 and 100% of the outer surface of each hybrid particle.

21. The material with hybrid particles as claimed in claim 20, the catalytic material covering an area between 10 and 100% of the outer surface of each hybrid particle.

22. The material with hybrid particles as claimed in claim 17, comprising a continuous layer, of a material different than the catalytic material encapsulating the PCM material.

23. The material with hybrid particles as claimed in claim 17, the catalytic material being in the form of a continuous layer encapsulating the PCM material.

24. The material with hybrid particles as claimed in claim 17, the catalytic material being in the form of a discontinuous layer partially covering the PCM material.

25. The material with hybrid particles as claimed in claim 17, the catalytic material being in the form of discrete particles dispersed on the surface or in the volume of each particle of MCP material.

26. The material with hybrid particles as claimed in claim 17, the catalytic material being in the form of an open structure in which at least one particle of PCM material is impregnated.

27. The material with hybrid particles as claimed in claim 17, the catalytic material being composed partly or completely with: Cu, Zn, Al, Cr, Ce, Zr, Pt, Pd, Ni, Ti, Si, and the corresponding oxides and nitrides.

28. A method of performing an exothermic or endothermic catalytic reaction comprising using a material with hybrid particles as claimed in claim 17.

29. A powder of material with hybrid particles as claimed in claim 17.

30. A method of manufacturing a material with hybrid particles, each consisting of a particle of a phase-change material interfaced with a catalytic material in solid form, according to which the following steps are carried out:
a/ production of a PCM material in powder form;
b/ deposition of a thin layer of a catalytic material continuously or discontinuously on the particles of PCM material.

31. The method of manufacture as claimed in claim 30, step b/ being carried out either by physical vapor deposition, by magnetron cathodic sputtering, or by chemical vapor deposition, or by a sol-gel technique, or by an electrodeposition technique, or by a liquid impregnation technique.

32. A method of manufacturing a material with hybrid particles, each consisting of a particle of a phase-change material interfaced with a catalytic material in solid form, according to which the following steps are carried out:
a1/ production of a PCM material in powder form;
a2/ production of a catalytic material in powder form;
b1/ mixing the powders of catalytic material and PCM material;
c1/ annealing the mixture above the phase change temperature of the PCM material;
d1/ grinding the annealed mixture.

33. A method of manufacturing a material with hybrid particles each consisting of a particle of a phase-change material interfaced with a catalytic material in solid form, according to which the following steps are carried out:
a′1/ production of a PCM material in powder form;
a′2/ production of a catalytic material in the form of an open structure;
b′/ impregnation of the open structure of the catalytic material with the PCM material dissolved or dispersed in an aqueous phase or a solvent;
c′/ annealing the impregnated structure above the phase change temperature of the PCM material and the temperature of evaporation of the water or solvent.

34. The method of manufacture as claimed in claim 30, wherein, once step a/ or all of production of the PCM material in powder form is completed, a step of deposition of a continuous layer, of a material different than the catalytic material, encapsulating the PCM material, is carried out.
